(19) United States
(12) Patent Application Publication
(10) Pub. No.: US 2007/0118944 A1
(43) Pub. Date: May 31, 2007

(54) Face shield

(76) Inventor: Gretchen Platt Stubenvoll, San Jose, CA (US)

Correspondence Address: Gretchen Platt Stubenvoll, 5178 Apennines Circle, San Jose, CA, 95138, US

(21) Appl. No.: 11/280,774

(22) Filed: Nov. 16, 2005

(51) Int. Cl. A61F 9/00

(52) U.S. Cl. 2/11

(57) ABSTRACT

A face shield for preventing an irritant from entering a person's eyes includes a shield member having front and back surfaces and a barrier member attached to the shield member which is engagable with a person's face. The face shield can also include a hand contact on the front surface adapted to receive at least a portion of the user's hand.

### BACKGROUND OF THE INVENTION

This invention relates to a face shield, and, particularly to a face shield for use during bathing, such as in hair washing, to prevent water, shampoo, conditioner and/or other irritants from entering a person's eyes.

Washing a person's hair, particularly a person of diminished, limited, or not fully developed capacity, such as a handicapped adult or a child, can be challenging especially when the person is nervous in the water, is afraid of getting water, shampoo and/or conditioner in his eyes, or is otherwise uncooperative. For example, oftentimes a child resists when the adult bathing the child attempts to wet the child's hair, apply shampoo or conditioner, and rinse the child's hair. The adult may attempt to wash the child's hair despite his protests. This response usually results in water, shampoo or conditioner getting into the child's eyes, and the child's hair not getting thoroughly cleaned. The end result is an unhappy child and a frustrated adult.

Bath time visors have long been used to keep water, shampoo and conditioner out of children's eyes when washing their hair. For example, a one piece foam device is available which includes a forward visor portion and a rearward integral strap portion. The visor portion deflects liquid from the child's eyes and the strap portion extends around the circumference of the child's head to attach the device to the child. This device often fails because it does not fit snuggly on younger children with smaller heads and it is difficult to thoroughly wash the hair held down and covered by the strap.

Alternative visor designs include an elastic strap, an adjustable strap or snaps for attaching the visor portion to the child's head. Although these visors are more securely fitted to the head, it remains difficult to thoroughly wash the hair held down and covered by such devices.

U.S. Pat. No. 5,564,154 by Cohn, III discloses a bathing mitt with a web member disposed between the thumb and forefinger for shielding a child's face from liquid. The adult inserts his entire hand into the mitt and extends the web member by spreading his thumb and forefinger. Then the adult places the bath mitt on the child's forehead such that the extended web member shields the child's eyes from liquid. Unfortunately, this device fails if the adult does not keep his thumb and forefinger properly extended. In that regard, the web buckles to form a gap, thus permitting liquid to run into the child's eyes. Furthermore, the adult will have difficulty removing his entire hand from this device, particularly when it becomes wet.

It is desirable to develop improved devices, systems and methods to shield at least a portion of a person's face (for example, from irritants during bathing).

### SUMMARY OF THE INVENTION

Thus, it is an object of the present invention to provide a face shield that limits or prevents water, shampoo, conditioner and/or other irritants from contacting or entering a person's (for example, a child's) eyes, nose and/or mouth. In several embodiments, the face shield is adapted to function generally independently of the person's head size.

It is another object of the present invention to provide a face shield for use during bathing that does not interfere with thorough washing, particularly of the hair.

It is another object of the present invention to provide a face shield that can be positioned and held in place by a user. As used herein, the term “user” refers to either a person utilizing the face shield on another or to a person utilizing the face shield on himself.

It is a further object of the present invention to provide a face shield that can easily be removed from a user's hand.

In one aspect the present invention provides a face shield for preventing an irritant from entering a person's eyes. The face shield includes: a shield member adapted to extend over at least a portion of the person's eyes and including a front surface, a back surface and a top edge; a barrier member attached to the shield member that is engagable with the person's face to provide contact between the barrier member and face; and a hand contact adapted to receive a portion of a hand. The shield member can be generally planar or curved. Likewise, the barrier member can be generally planar or curved. The barrier member can, for example, be attached to the back surface of the shield member or to the top edge of the shield member. The barrier member can be compressible to form a generally sealing engagement with at least a portion of the person's face. The shield member can be flexible. Likewise, the barrier member can be flexible. The shield member and/or the barrier member an also be transparent. The shield member can further include an outer edge; and a protective edging on at least a portion of the outer edge.

The hand contact can, for example, include at least one friction hold adapted to receive at least one finger of the hand. The friction hold can be flexible. The hand contact can also or alternatively include at least one projection extending outward from the front surface adapted to receive at least one finger. The hand contact can also or alternatively include a hand attachment extending outward from the front surface adapted to attach the face shield to the hand. The hand attachment can be removeably attached to the front surface. In several embodiments, the hand attachment includes at least one finger hook, finger pocket, finger strap, finger recess or finger loop on the front surface, each of which is adapted to receive at least one finger of a hand. The at least one finger hook, finger pocket, finger strap, finger recess or finger loop can be located generally opposite the barrier member. The hand attachment also or alternatively includes at least one hook, strap or loop extending outward from the front surface that is adapted to receive the hand.

In another aspect, the present invention provides a face shield for preventing an irritant from entering a person's eyes. The face shield includes: a shield member adapted to extend over at least a portion of the person's eyes, the shield member including a front surface; a barrier member attached to the shield member that is engagable with the person's face to provide contact between the barrier member and face; a hand contact on the front surface for receiving at least a portion of a hand and including at least one friction hold proximately located opposite the barrier member for receiving at least one finger of the hand; and a hand attachment extending outward from the front surface for attaching the face shield to the hand.

In still a further aspect, the present invention provides a method of shielding a person's face to prevent an irritant from entering a person's eyes including: grasping a shield comprising a barrier member attached to the shield and placing the shield in proximity to the person's face; and applying force to the shield to engage the barrier member with the person's face to provide contact between the barrier member and the person's face. The barrier member can be compressed against the person's face. The person can, for example, be a child being bathed, and the shield can be grasped by a person other than the child.

The face shields of the present invention extend over at least a portion of a child's or other person's eyes during use. The barrier member extends from the shield and is engagable with the child's or other person's face to provide contact between the shield member and face. The hand contact is adapted to receive at least a portion of the user's hand so that the user can readily and easily apply a compressive force to the shield/barrier member. The face shields of the present invention do not require any type of attachment member extending around the head of the child or other person in connection with whom the device is used, thereby provide ready access to the child's or other person's hair.

### BRIEF DESCRIPTION OF THE DRAWINGS

FIG. 1 is a front view of an embodiment of the invention;

FIG. 2A is a back view of the embodiment shown in FIG. 1;

FIG. 2B is a cross-sectional view of the embodiment shown in FIG. 1;

FIG. 3A is a front view of another embodiment of the invention;

FIG. 3B is a cross-sectional view of the embodiment shown in FIG. 3A;.

FIG. 4 is a front view of another embodiment of the invention;

FIG. 5 is a back view of the embodiment shown in FIG. 4;

FIG. 6 is a front view of another embodiment of the invention;

FIG. 7 is a cross-sectional view of the embodiment shown in FIG. 6;

FIG. 8A is a front view of another embodiment of the invention;

FIG. 8B is a cross-sectional view of the embodiment shown in FIG. 8A;

FIG. 9A is a front view of another embodiment of the invention;

FIG. 9B is a cross-sectional view of the embodiment shown in FIG. 9A;

FIG. 10 is a front view of another embodiment of the invention;

FIG. 11 is a cross-sectional view of the embodiment shown in FIG. 10;

FIG. 12 is a front view of another embodiment of the invention;

FIG. 13 is a front view of another embodiment of the invention;

FIG. 14 is a front view of another embodiment of the invention;

FIG. 15 is a cross-sectional view of the embodiment shown in FIG. 14;

FIG. 16A is a front view of another embodiment of the invention;

FIG. 16B is a cross-sectional view of the embodiment shown in FIG. 16A;

FIG. 17 is a front view of another embodiment of the invention;

FIG. 18 is a front view of another embodiment of the invention;

FIG. 19 is a cross-sectional view of the embodiment shown in FIG. 18;

FIG. 20 is a front view of another embodiment of the invention;

FIG. 21 is a cross-sectional view of the embodiment shown in FIG. 20;

FIG. 22 is a front view of another embodiment of the invention; and

FIG. 23 is a cross-sectional view of the embodiment shown in FIG. 22.

### DETAILED DESCRIPTION OF THE INVENTION

Several representative embodiments of face shields of the present invention are discussed herein in connection with use thereof in bathing a person, and, particularly, a child. One skilled in the art, however, appreciates that the face shields of the present invention can be used in a wide variety of situations in addition to bathing as well as in connection with any person to shield at least a portion of the person's face. For example, the face shields of the present invention may also be used during hairstyling to prevent hair spray from entering a person's eyes.

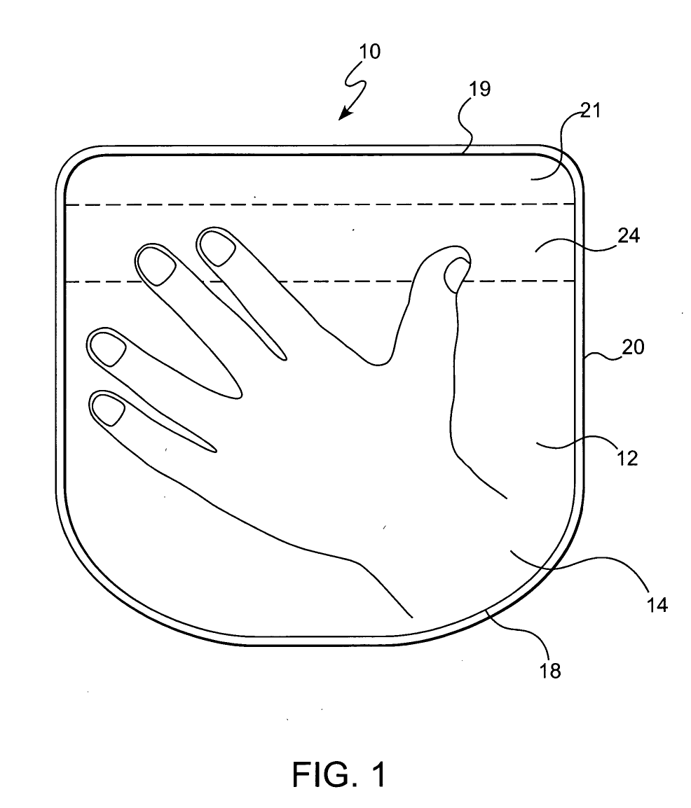

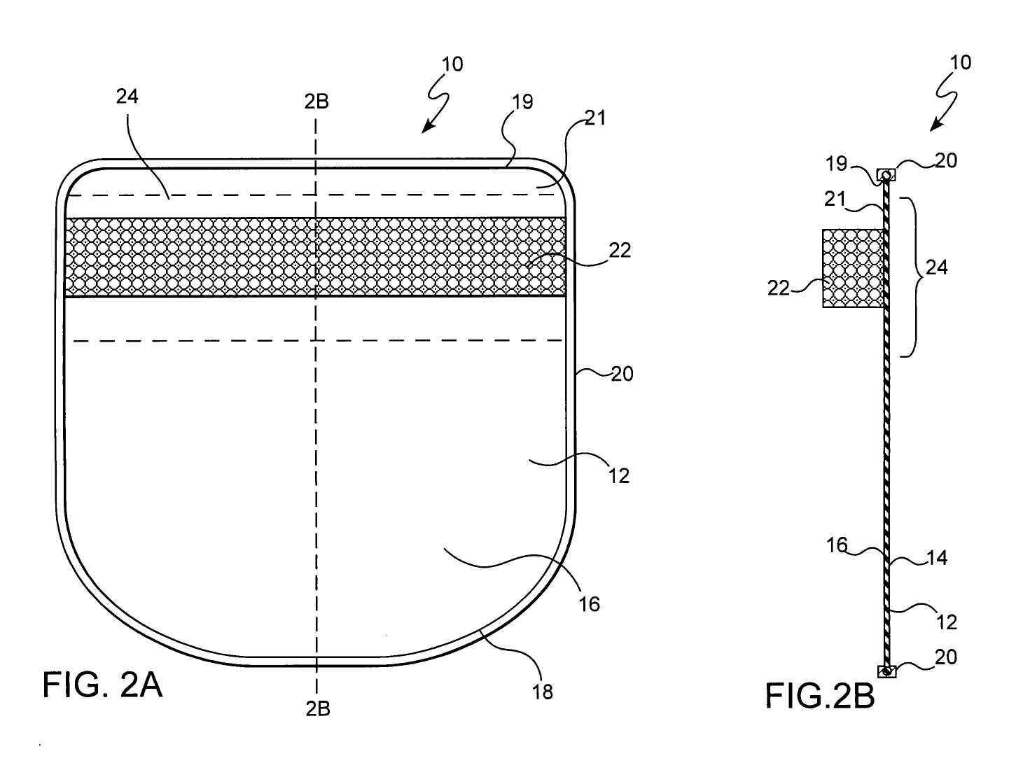

An embodiment of the face shield 10 of the present invention is shown in FIGS. 1 through 2B. The face shield 10 includes a shield member 12 which covers or shields at least a portion of a person's face (for example, a child's eyes) when the shield 10 is positioned on his face. Face shield 10 can also extend downward to cover or shield the child's nose or mouth. The shield member 12 includes a front surface 14, back surface 16, outer edge 18 and top edge 19. Protective edging 20 can also be provided around the perimeter of outer edge 18 and/or top edge 19 to reduce any likelihood of injury to the child or adult bathing the child from contact with outer edge 18 and top edge 19. The shield member 12 can, for example, be made of any liquid impervious or repellent material, including, but not limited to, polymeric materials such as plastic, foam or vinyl. Shield member 12 can be rigid or flexible, opaque or transparent. Shield member 12 can be generally planar or curved in shape. In a preferred embodiment, the shield member 12 is flexible and transparent. Examples of suitable materials for shield member 12 include polyethylene, polypropylene and/or polycarbonate.

The face shield 10 of the present invention further includes a barrier member 22 attached the shield member 12 (for example, to the back surface of shield member 12) at or below the top edge 19 by any suitable attachment mechanism or means, such as stitching, a waterproof fastener or an adhesive as known in the attachment arts. As shown in FIGS. 1 through 2B, when barrier member 22 is attached below top edge 19, a liquid deflector 21 is formed at the back surface 16 of shield member 12. When face shield 10 includes a deflector 21, additional liquid can be prevented from splashing over top edge 19 and onto front surface 14 of shield member 12. The transparent shield member 12 preferably allows the child to watch the adult while, for example, having his hair washed.

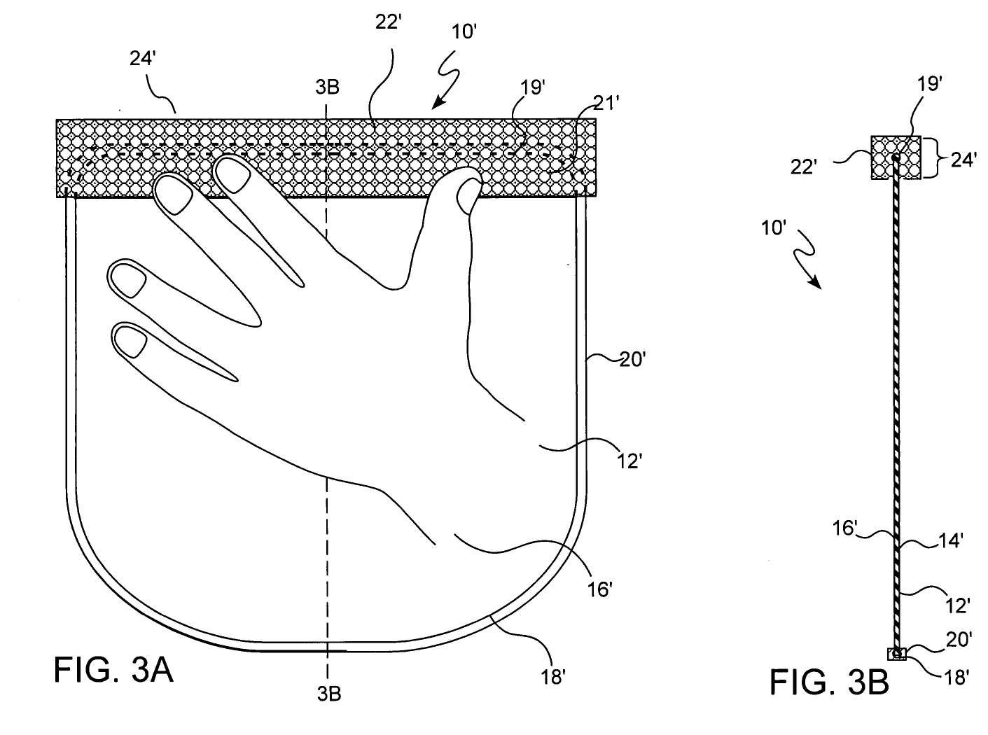

FIGS. 3A and 3B illustrate an alternative embodiment wherein a barrier member 22′ is attached to top edge 19′ of face shield 10′. In general, the operation of face shield 10′ is very similar to the operation of face shield 10 and like components are numbered similarly with the addition of a designation “′” thereto.

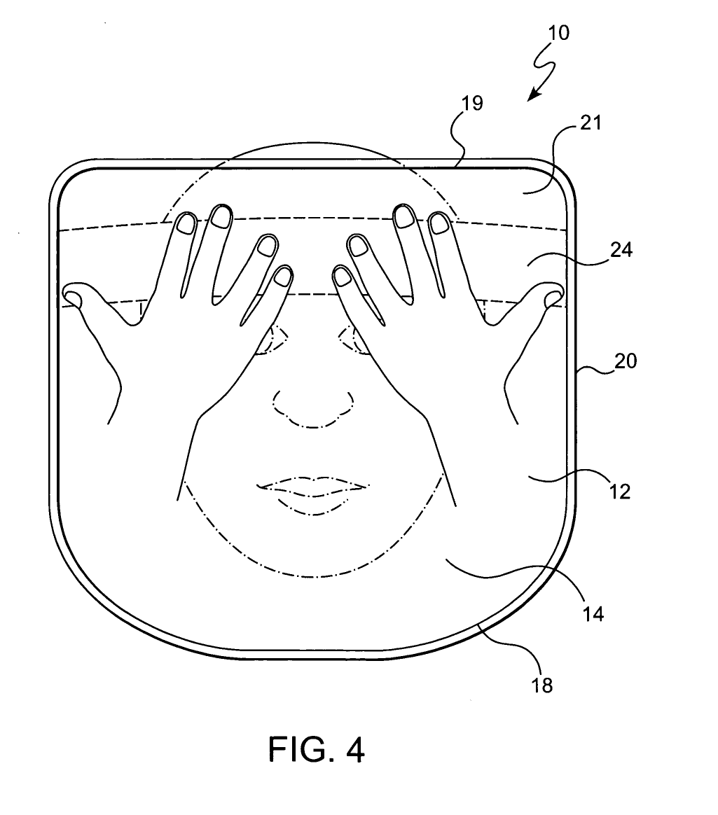

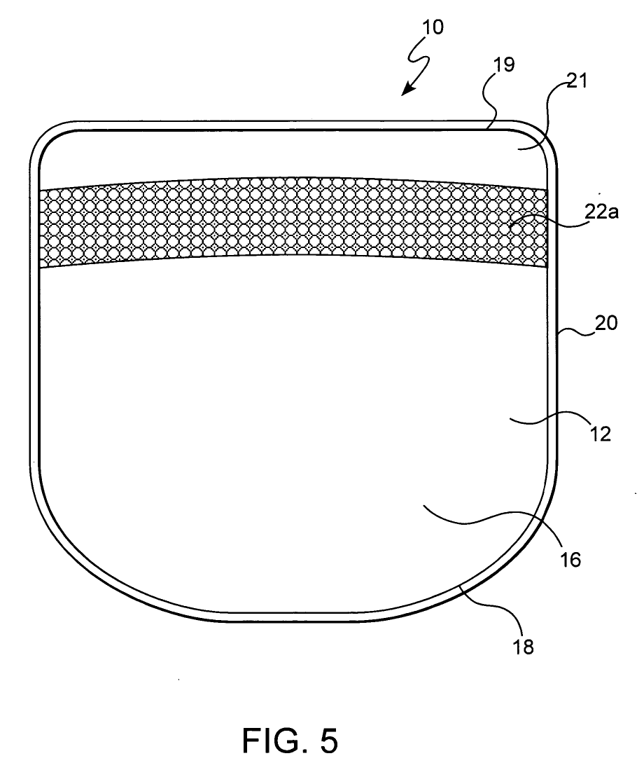

Referring back to FIGS. 1 through 2B, barrier member 22 preferably extends horizontally across the width of the shield member 12 at least a distance equal to the spacing of the eyes. In another embodiment, as shown in FIGS. 4 and 5, barrier member 22a is curved downward at its outer, lateral edges to prevent liquid from entering the top and sides of the person's eyes. In this embodiment, barrier member 22a can provide a continuous barrier to liquid across the person's forehead and down along the sides of his face. Barrier members 22 and 22a can, for example, be made of any liquid resistant or liquid absorbent material such as foam or sponge and can also be flexible, compressible or resilient to, for example, facilitate conformation to the person's face and/or sealing to the person's face as well as to enhance the comfort of the person. Suitable materials for barrier member 22a include, but are not limited to, polymeric materials such as foam rubbers (for example, polyurethane rubbers) and various elastomers.

Referring again, for example, to FIGS. 1, 2A, 3A and 4, face shield 10 can also include one or more hand contacts adapted to receive at least a portion of the user's hand and facilitate the application of force to shield 12 to compress barrier member 22 into contact with the child's face. Such a hand contact can, for example, include a hand contact area 24 or 24′ located on the front surface 14 of shield 12. In the embodiments illustrated in FIGS. 1, 2A, 3A and 4, hand contact area 24 is positioned generally opposite the barrier member 22. Hand contact area 24 can be coextensive in area with barrier member 22, cover a larger area that barrier member 22 or cover a smaller area the barrier member 22. Moreover, hand contact area 24 need not be positioned opposite barrier member 22. As shown, for example, in FIG. 1, hand contact area 24 is adapted (for example, textured) to receive at least a portion of the user's hand, such as a thumb and forefinger, which are used to apply force to shield 12 and to, thereby, compress barrier member 22 into contact with the child's face. In that regard, barrier member 22 of face shield 10 is held in contact with the child's face and compressed via application of force to shield member 12 to prevent liquid from, for example, entering the child's eyes. Alternatively, FIG. 4 shows the hand contact area 24 can receive the child's hands which compress barrier member 22 or 22a onto the child's face.

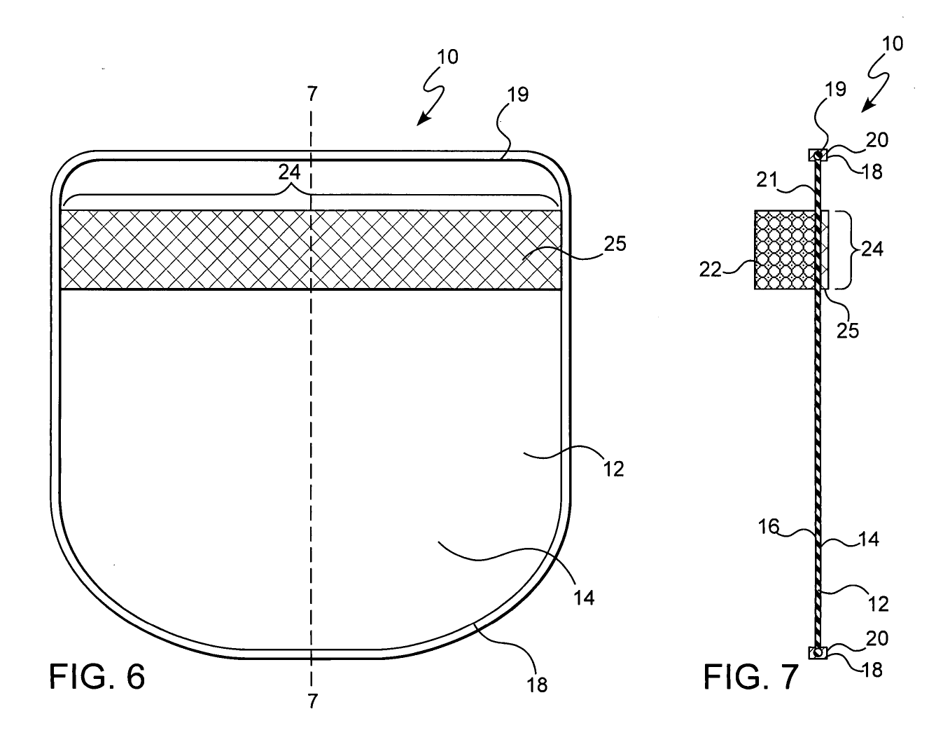

As illustrated in FIGS. 6 and 7, the hand contact area24 can include at least one friction hold 25 to prevent the user's fingers from slipping from the hand contact area 24. The friction hold 25 can, for example, include a textured material known in the art, which can be a flexible material. Front surface 14 of shield 12 can be adapted or textured (for example, knurled) in one or more regions of hand contact area 24 to form friction hold 25. Alternatively, the entirety of front surface 14 of shield member 12 can be textured or otherwise adapted to facilitate grasping of face shield 10 and application of compressive force to barrier member 22. In such an embodiment, the hand contact area would be coextensive with shield member 12. As shown in FIG. 6, friction hold 25 may extend the entire width of the front surface 14 proximately opposite barrier member 22. Alternatively, friction hold 25 can be formed as one or more areas of textured material/surface which can, for example, be located generally opposite barrier member 22.

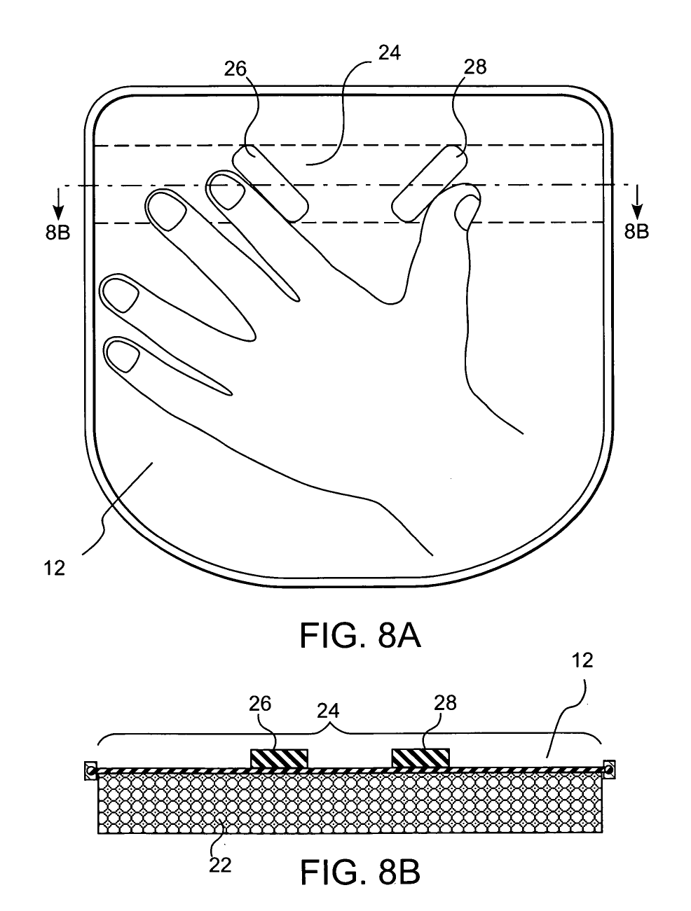

In another embodiment, as shown in FIGS. 8A and 8B, in addition to or in the alternative to hand contact area 24, shield 12 can include a hand contact including one or more abutment members such as protrusions, projections, flanges or nubs 26 and 28 protruding or projecting outwardly from the front surface 14 of shield member 12 for receiving in abutting engagement one or more fingers of the user's hand. When the user's fingers engage projections 26 and 28, the user can hold on to face shield 10 and compress the barrier member 22 onto the child's face.

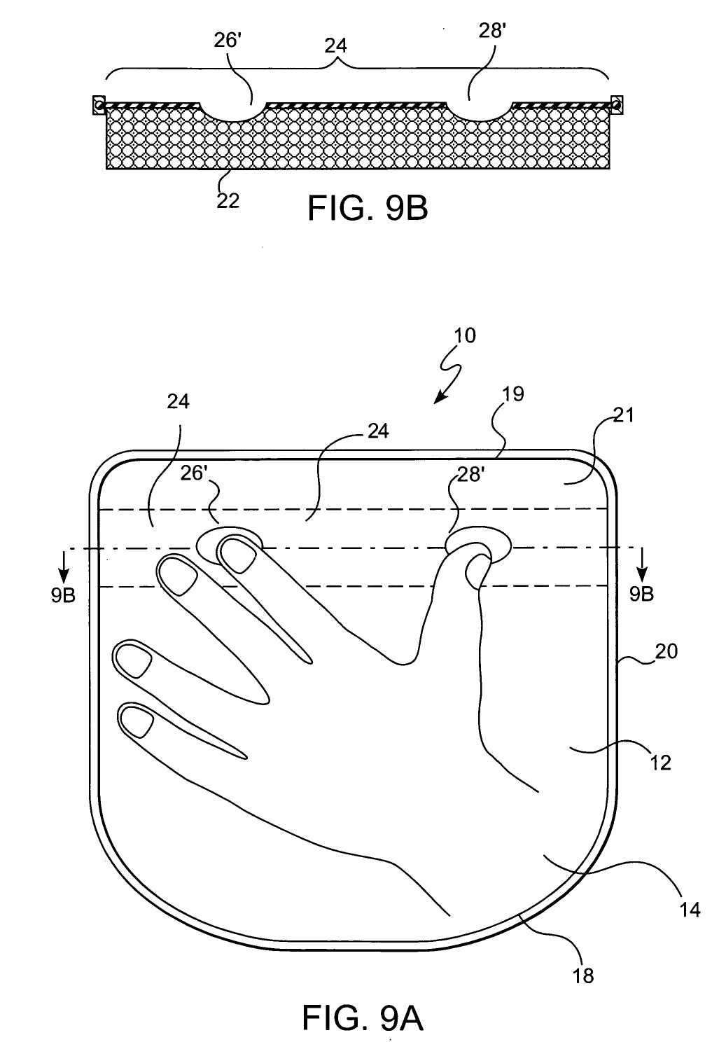

In the embodiment illustrated in FIGS. 9A and 9B, shield 12 additionally or alternatively includes one or more hand contact abutment members formed as recesses 26′ and 28′ extending inwardly from the front surface 14 of shield member 12 for receiving in abutting engagement one or more fingers of the user's hand. In the embodiment of FIGS. 9A and 9B, recesses 26′ and 28′ extend through shield member 12 in the area of friction hold 25 and partially into barrier member 22.

The face shields of FIGS. 1 through 9B are held in place on a person's face, such as a child's face, by the user, previously defined as either a person utilizing the face shield on another or to a person utilizing the face shield on himself. For example, a child as the user may position the face shield 10 onto his face by placing the barrier member 22 on his forehead, brow line, or directly covering his eyes. Then using one or both hands, the child positions his fingers or palms on the hand contact area 24 and/or other hand contact of the present invention, and applies suitable force to compress the barrier member 22 onto his face. Barrier member 22 thereby creates a generally sealing contact between the face shield 10 and the child's face.

Alternatively, an adult, as the user, may position the face shield 10 onto the child's face by placing the barrier member 22 on the child's forehead, brow line, or covering his eyes. Then using one hand, the adult positions at least one finger and thumb, or his palm on to the hand contact area 24 and/or other hand contact of the present invention and applies force to compress barrier member 22 onto or into contact with the child's face. Again, barrier member 22 can create a sealing engagement between the face shield 10 and the child's face.

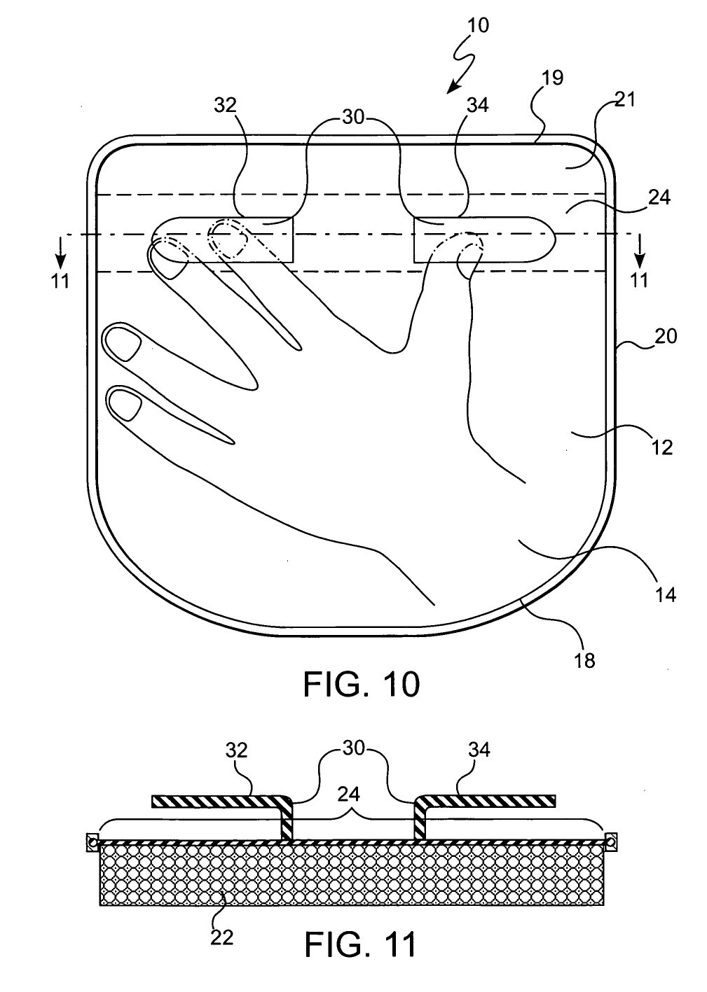

In several other embodiments of the present invention, as shown, for example, in FIGS. 10 through 23, face shield 10 of the present invention further includes a hand contact including a finger and/or hand attachment 30 for attaching the face shield 10 to the user's hand. A function of the hand attachment 30 is to allow the user to intermittently remove the face shield 10 from a person's face while still holding onto the face shield 10. Thus, the user will not have to repeatedly pick up the face shield 10 when the person's eyes need to be shielded again.

FIGS. 10 and 11 show hand attachment 30 may include at least one finger hook for receiving at least one finger of the user's hand. In the embodiment illustrated in FIGS. 10 and 11, hand attachment 30 includes two finger hooks or handles 32 and 34 for receiving the user's thumb and at least one finger. Finger hooks 32 and 34 can, for example, be made of a rigid or semi-rigid material, including, but not limited to, a polymeric materials such as a plastic or a foam, and can be attached to the front surface 14 of shield member 12 by any suitable attachment mechanism or means, such as a waterproof fastener or adhesive. Finger hooks 32 and 34 can, for example, extend outward from the hand contact area 24 at a position generally opposite the barrier member 22 to, for example, facilitate application of compressive force to barrier member 22. The user places his forefinger and thumb onto the hand contact area 24 while inserting his forefinger under hook 32 and inserting his thumb under hook 34.

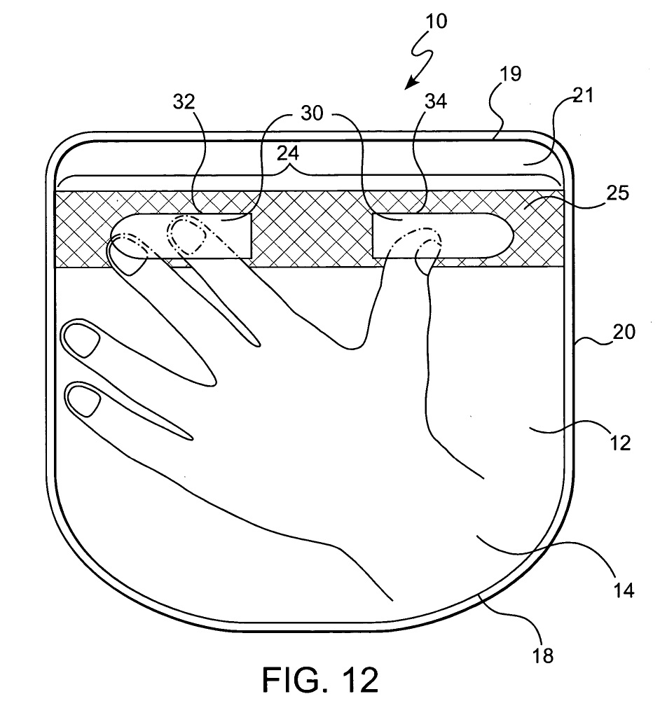

As shown in FIG. 12, hand contact area 24 may also include friction hold 25 as previously described in regards to FIG. 8. The user may compress barrier member 22 to the child's forehead by pressing his forefinger and thumb on the hand contact area 24. When the user needs to remove the face shield 10 from the person's face, the user releases the compression of his forefinger and thumb, and separates his hand from the person's head. The face shield 10 remains attached to the user's forefinger and thumb via finger hooks 32 and 34.

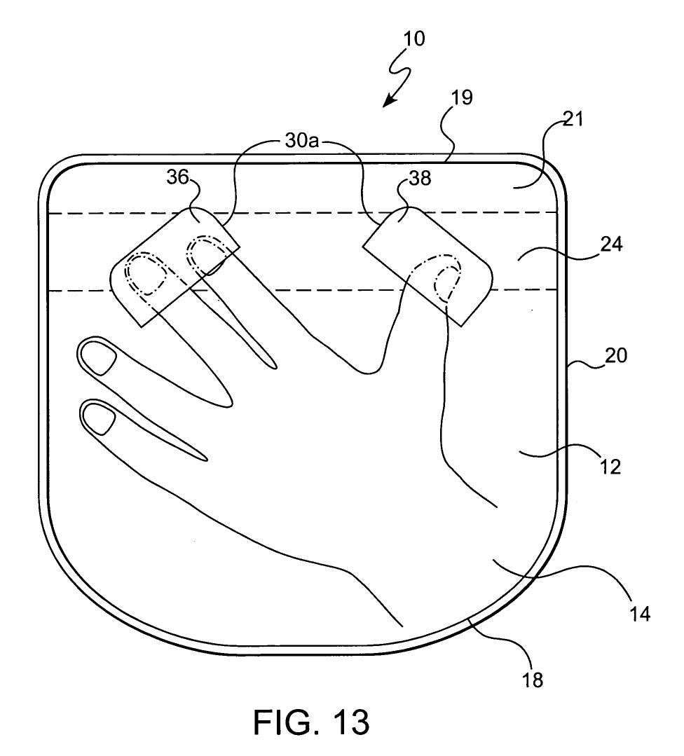

Another embodiment of a hand attachment 30a is shown in FIG. 13. Hand attachment 30a includes at least one finger pocket for receiving at least one finger or thumb or the user's hand. Similar to the finger hooks 32 and 34 described above, finger pockets 36 and 38 can, for example, be made of a rigid or semi-rigid material such as plastic or foam. Alternatively, for added comfort, finger pockets 36 and 38 may be made of a softer material such as fabric or canvas. In the illustrated embodiment, finger pockets 36 and 38 extend outward from the hand contact area 24 generally opposite the barrier member 22, and are attached to the front surface 14 of shield member 12 by any suitable attachment mechanism or means, such as stitching, a waterproof fastener or adhesive. However, as with a number of other hand contacts of the present invention, shield 10 can include finger pockets 36 and 38 without hand contact area 24. In the illustrated embodiment, the user places his first two fingers and thumb onto the hand contact area 24 while inserting his fingers into finger pocket 36 and inserting his thumb into finger pocket 38. Then, the user may compress barrier member 22 to the person's forehead by pressing his fingers and thumb on the hand contact area 24. When the user needs to remove the face shield 10 from the person's face, the user releases the compression of his fingers and thumb, and separates his hand from the person's head. The face shield 10 remains attached to the user's forefinger and thumb via finger pockets 36 and 38.

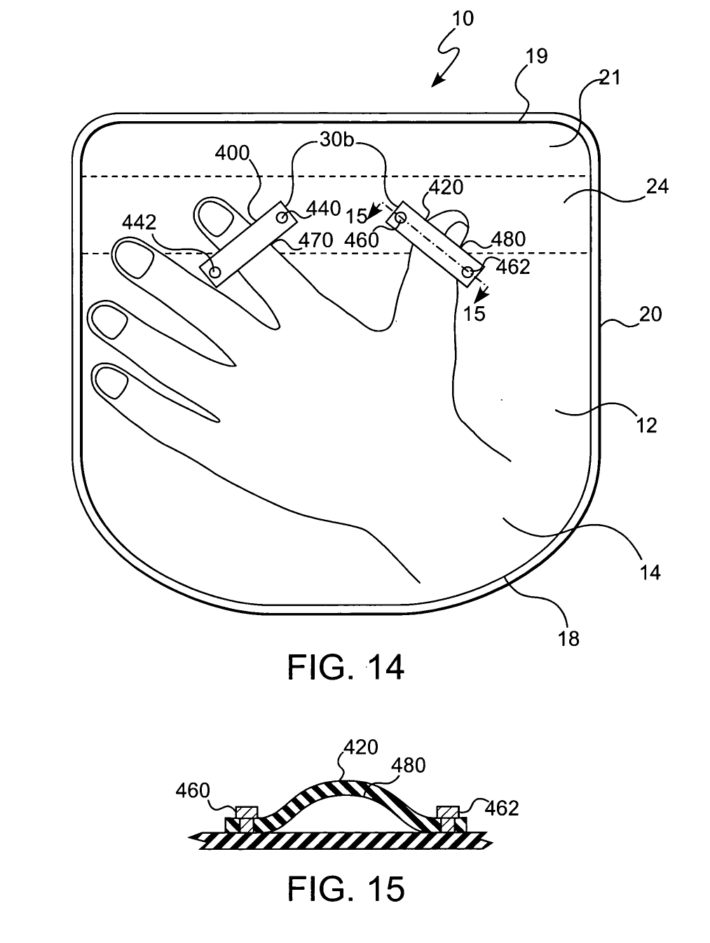

Similar to the finger pockets 36 and 38 described above, a hand attachment 30b can alternatively include finger straps 400 and 420, as shown in FIGS. 14 and 15. Finger straps 400 and 420 can, for example, be made of any suitable material such as plastic, foam or fabric. Finger straps 400 and 420 extend outward from the hand contact area 24 proximately opposite the barrier member 22, and are attached to the front surface 14 of shield member 12 by any suitable attachment mechanism or.means. In this embodiment, finger straps are attached at their respective ends to the front surface 14 by waterproof fasteners 440, 442, 460 and 462. Finger straps 400 and 420 can be removeably attached to the front surface 14 in the case that each of fasteners 440, 442, 460 and 462 is a removable fastener such as a snap or similar device. Opening 470 for receiving at least one of the user's fingers is formed by finger strap 400 and front surface 14. Opening 480 for receiving the user's thumb is formed by finger strap 420 and front surface 14. The user places his forefinger and thumb onto the hand contact area 24 while inserting his forefinger into opening 470 and inserting his thumb into opening 480. Then, similar to the device described in reference to FIG. 13, the user may compress barrier member 22 to the child's forehead by pressing his forefinger and thumb on the hand contact area 24. Face shield 10 remains attached to the user's hand via finger straps 400 and 420.

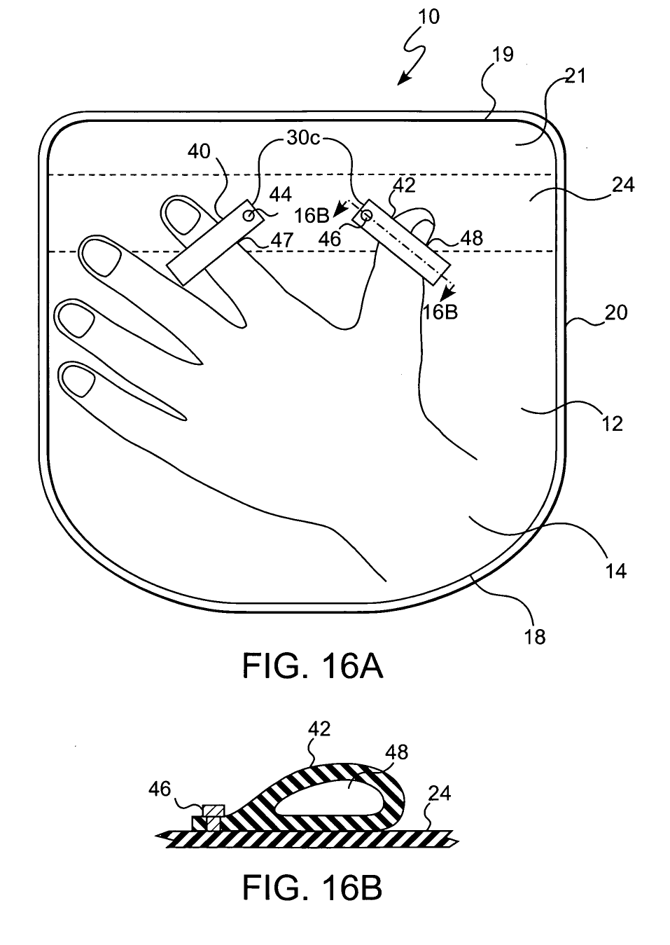

FIGS. 16A and 16B show another embodiment of a hand attachment 30c. In this configuration hand attachment, 30c includes finger loops 40 and 42 made of any plastic, foam or fabric. Finger loops 40 and 42 extend outward from the hand contact area 24 generally opposite the barrier member 22, and are attached to the front surface 14 of shield member 12 by any suitable attachment means. As shown in FIGS. 16A and 16B, finger loops 40 and 42 are attached to the front surface 14 by pivots 44 and 46, respectively. Pivots 44 and 46 allow loops 40 and 42 to rotate upon the front surface 14 to accommodate the span between the user's thumb and forefinger when the user inserts his forefinger into finger loop 40 via a first opening 47 and his thumb into finger loop 42 via a second opening 48. Finger loops 40 and 42 can be removeably attached to front surface 14 in the case that each of pivot 44 and 46 is a removable fastener such as a snap or similar device. Operation of this embodiment is similar to that of the embodiments shown in FIGS. 10 through 15.

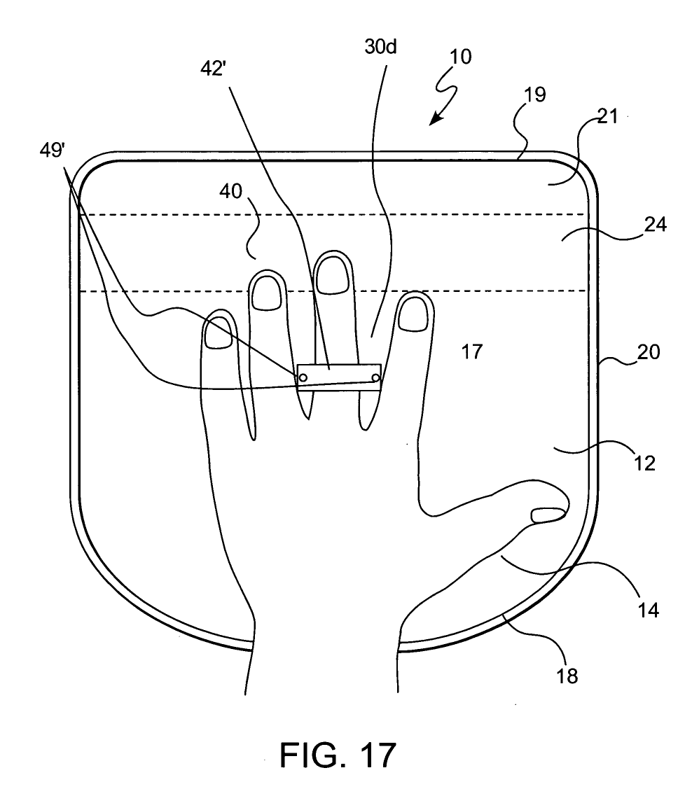

FIG. 17 illustrates a further embodiment of a hand attachment 30d including a single finger loops 42′ made of, for example, a plastic, fabric. In the illustrated embodiment, finger loops 42′ extend outward from the hand contact area 24 below (in the orientation of FIG. 17) the position of barrier member 22. Finger loop 42′ can, for example, be attached to the front surface 14 of shield member 12 by fasteners 49′.

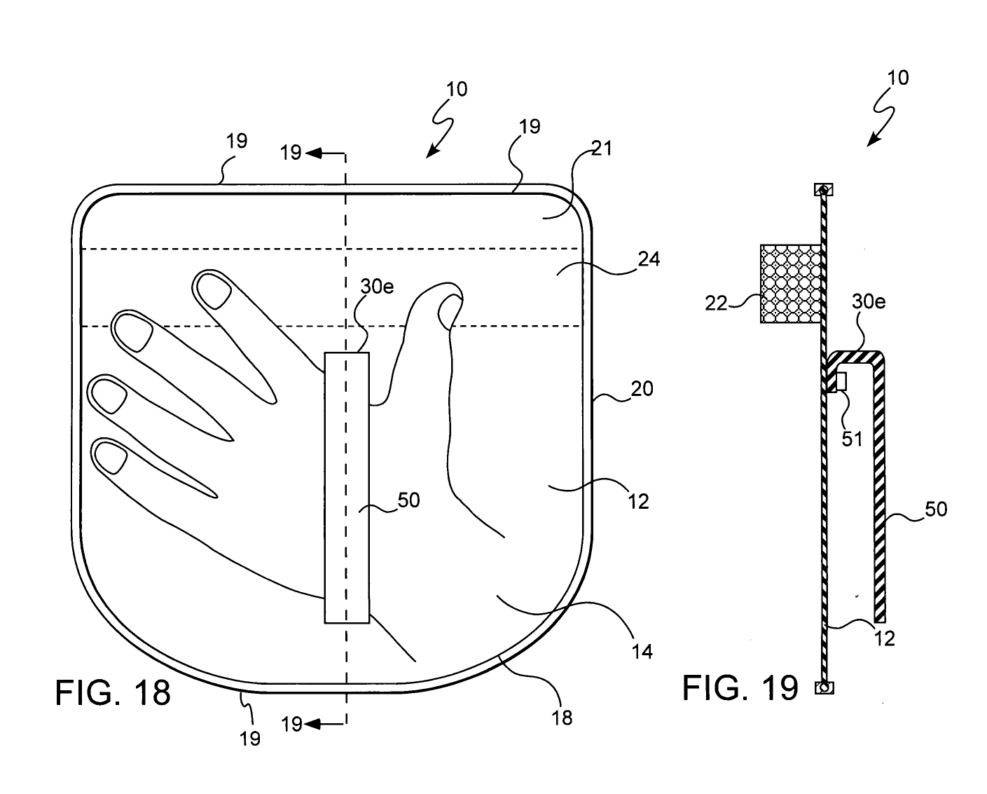

The hand attachments of the present invention can also include a device which attaches the shield member 12 to the palm or the back of the user's hand. As shown in FIGS. 18 and 19, hand attachment 30e can include a hand hook or handle 50 made, for example, of a rigid or semi-rigid material such as plastic or foam. Hand hook 50 extends outward from front surface 14 and can be attached thereto by any suitable mechanism or means, such as a waterproof fastener 51 or adhesive. Hand hook 50 can be removeably attached to front surface 14 in the case that fastener 51 is a removable fastener such as a snap or similar device. Hand hook 50 can, for example, be located or positioned near the center line of top edge 19 and at or below the hand contact area 24. The user places his hand under hand hook 50 and positions at least his forefinger and thumb onto hand contact are 24. Then, the user may compress barrier member 22 to a person's forehead by pressing his finger and thumb on the hand contact area 24. When the user needs to remove the face shield 10 from the person's face, the user releases the compression of his fingers and thumb, and separates his hand from the person's head. The face shield 10 remains attached to the user's hand via hand hook 50.

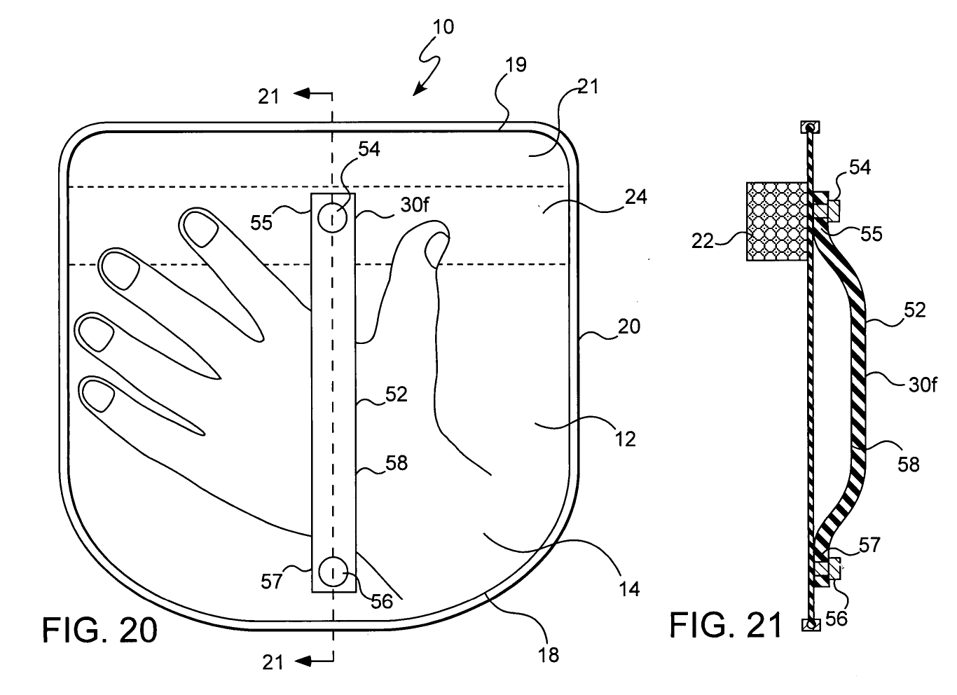

Referring now to FIGS. 20 and 21, hand attachment 30f can, for example, also include a hand strap 52 made of any suitable material such as plastic, foam or fabric. Hand strap 52 extends outward from front surface 14 and includes a first end 55 and a second end 57. Each end 55 and 57 is attached to the front surface 14 by any suitable mechanism or means, such as stitching, waterproof fasteners 54 and 56, or adhesive. Hand strap 52 is removeably attached to the front surface 14 in the case that each fastener 54 and 56 is a removable fastener such as a snap or similar device. Opening 58 for receiving the user's hand is formed by hand strap 52 and front surface 14. Hand strap 52 can, for example, be located approximately at the center line of top edge 19 and at or below the hand contact area 24. The user places his hand into opening 57 such that the hand strap 52 extends over the back of his hand. Then the user positions at least his forefinger and thumb onto hand contact are 24. Operation of this embodiment of the invention is similar to that of the device shown in FIGS. 23 and 24, and face shield 10 remains attached to the user's hand via hand strap 52.

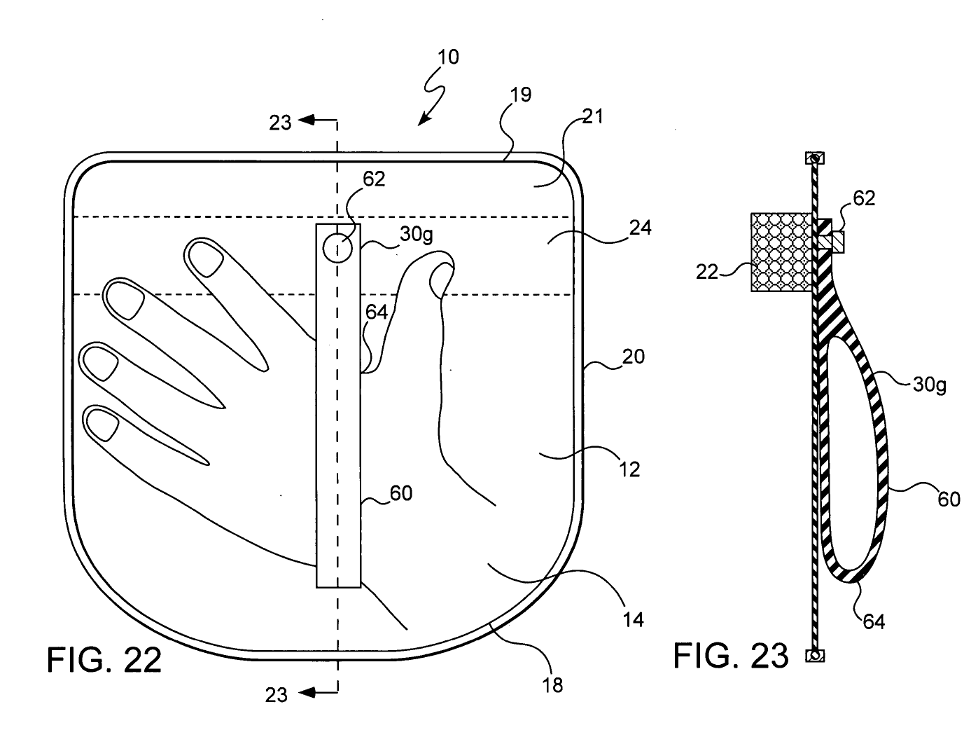

Another embodiment of the present invention is shown in FIGS. 22 and 23. In this embodiment, hand attachment 30g includes a hand loop 60 made of any suitable material such as plastic, foam or fabric. As shown in FIGS. 27 and 28, hand loop 60 extends outward from front surface 14 and can be located generally at the center line of top edge 19 and at or below the hand contact area 24. Hand loop 60 can be attached to front surface 14 by any suitable mechanism or means including, for example, stitching, waterproof fastener 62, or adhesive. When fastener 62 is a pivot, as shown in FIGS. 27 and 28, it permits movement of the hand loop 60 relative to the front surface 14. When fastener 62 is a removable fastener such as a snap, hand loop 60 is removeably attached to front surface 14. Hand loop 60 includes opening 64 for receiving the user's hand.

In this embodiment, the user places his hand into opening 64 such that the hand loop 60 extends over the back and palm of his hand. Then the user positions at least his forefinger and thumb onto hand contact are 24. Because hand loop 60 pivots via fastener 62, the user's can more easily position his forefinger and thumb onto the hand contact area 24. For example, the user may find the present invention more comfortable to use when his forefinger is placed on to the hand contact area 24 closer to the top edge 19 and his thumb is placed on the hand contact area 24 further from the top edge 19. Operation of this embodiment is similar to that of the devices shown in FIGS. 19 through 24, and face shield 10 remains attached to the user's hand via hand loop 60.

When a child or an adult is holding the face shield 10 in place by any of the embodiments described above, the adult can, for example, wet the child's hair, apply shampoo or conditioner, and rinse the child's hair, all without any liquid entering the child's eyes. Any liquid coming in contact with barrier member 22 is deflected away from the eyes to the sides of the child's face. When made of an absorbent material, barrier member 22 also absorbs some of the liquid, thus further keeping the child's eyes clear. Front surface 14 of shield member 12 prevents any liquid from entering the child's eyes, nose and mouth by deflecting liquid toward the outer edge 18 of shield member 12.

The foregoing description and accompanying drawings set forth embodiment, including preferred embodiments, of the invention at the present time. Various modifications, additions and alternative designs will, of course, become apparent to those skilled in the art in light of the foregoing teachings without departing from the scope of the invention. The scope of the invention is indicated by the following claims rather than by the foregoing description. All changes and variations that fall within the meaning and range of equivalency of the claims are to be embraced within their scope.

1. A face shield for preventing an irritant from entering a person's eyes, the face shield comprising:
a flexible shield member adapted to extend over at least a portion of the person's eyes, the shield member including a front surface, a back surface and a top edge;
a liquid resistant flexible and compressible barrier member attached to the shield member and projecting outward from the back surface of the shield member that is engagable with the forehead of the person's face to provide sealing contact between the barrier member and the forehead of the face; and
a hand contact positioned on the front surface of the shield member for receiving at least a portion of a hand such that at least one finger of the hand is positioned proximatelv opposite the barrier member to compress the barrier member onto the person's forehead.

2. The face shield of claim 1 wherein the shield member is curved.

3. The face shield of claim 1 wherein the barrier member is attached to the back surface of the shield member.

4. The face shield of claim 1 wherein the barrier member is attached to the top edge of the shield member.

5. (canceled)

6. The face shield of claim 1 wherein the barrier member is curved.

7. The face shield of claim 1 wherein the hand contact includes at least one friction hold adapted to receive at least one finger of the hand.

8. (canceled)

9. (canceled)

10. The face shield of claim 1 wherein the shield member is transparent.

11. The face shield of claim 1 wherein the shield member further includes an outer edge; and a protective edging on at least a portion of the outer edge.

12. The face shield of claim 7 wherein the friction hold is flexible.

13. The face shield of claim 1 wherein the hand contact comprises at least one projection extending outward from the front surface adapted to receive at least one finger.

14. The face shield of claim 1 wherein the hand contact comprises a hand attachment extending outward from the front surface adapted to attach the face shield to the hand.

15. The face shield of claim 14 wherein the hand attachment is removeably attached to the front surface.

16. The face shield of claim 14 wherein the hand attachment comprises at least one finger hook finger pocket, finger strap, finger recess or finger loop on the front surface and is adapted to receive at least one finger of a hand.

17. The face shield of claim 16 wherein the at least one finger hook, finger pocket, finger strap, finger recess or finger loop is located generally opposite the barrier member.

18. The face shield of claim 14 wherein the hand attachment includes at least one hook, strap or loop extending outward from the front surface that is adapted to receive the hand.

19. A face shield for preventing an irritant from entering a person's eyes, the face shield comprising:
a flexible shield member adapted to extend over at least a portion of the person's eyes, the shield member including a front surface and a back surface;
a liquid resistant, flexible and compressible barrier member attached to the shield member and projecting outward from the back surface of the shield member that is engagable with the forehead of the person's face to provide sealing contact between the barrier member and the forehead of the face;
a hand contact positioned on the front surface of the shield member for receiving at least a portion of a hand and including at least one friction hold proximately located opposite the barrier member for receiving at least one finger of the hand such that the finger is positioned proximately opposite the barrier member to compress the barrier member onto the person's forehead; and
a hand attachment positioned on and extending outward from the front surface of the shield for attaching the face shield to the hand.

20. (canceled)

21. (canceled)

22. (canceled)

23. A face shield for preventing an irritant from entering a person's eyes, the face shield comprising:
a flexible shield member adapted to extend over at least a portion of the person's eyes, the shield member including a front surface and a back surface;
a liquid resistant, flexible and compressible barrier member attached to the shield member and projecting outward from the back surface of the shield member that is engagable with the forehead of the person's face to provide sealing contact between the barrier member and the forehead of the face;
a hand contact positioned on the front surface of the shield member for receiving a portion of a hand such that at least one finger of the hand is proximately positioned opposite the barrier member to compress the barrier member onto the person's forehead; and
a hand attachment positioned on and extending outward from the front surface of the shield for attaching the face shield to the hand.
